(19) United States
(12) Patent Application Publication
(10) Pub. No.: US 2010/0262045 A1
(43) Pub. Date: Oct. 14, 2010

(54) PATIENT MONITORING METHOD AND SYSTEM

(75) Inventors: David William Heaton, York (GB); Joy Margaret Revie, Boroughbridge (GB); Ian Crawford Revie, Boroughbridge (GB)

Correspondence Address: CONLEY ROSE, P.C.;David A. Rose, P. O. BOX 3267, HOUSTON, TX, 77253-3267, US

(73) Assignee: Activ4Life Healthcare Technologies Limited, Boroughbridge (GB)

(21) Appl. No.: 12/663,816

(22) PCT Filed: Jun. 6, 2008
(86) PCT No.: PCT/GB08/01989

§ 371 Date: Jun. 17, 2010

(30) Foreign Application Priority Data
Jun. 9, 2007 (GB) 0711223.8
Mar. 13, 2008 (GB) 0804671.6

(51) Int. Cl. A61B 5/11

(52) U.S. Cl. 600/595

(57) ABSTRACT

The present invention relates to patient monitoring methods and patient monitoring systems for monitoring at least one aspect of a subject patient's motion alone or with one or more additional patient subject related measurements. The patient monitoring method and system can be a useful clinical tool in the monitoring of patients, and in particular, although not exclusively orthopaedic patients, both pre-treatment and post treatment.

### FIELD OF THE INVENTION

The present invention relates to patient monitoring methods and patient monitoring systems for monitoring at least one aspect of a subject patient's motion alone or with one or more additional patient subject related measurements.

### BACKGROUND TO THE INVENTION

A person's activity is affected by a number of factors, including their health. Clearly, for a number of diseases, as the disease progresses in a person that person may become progressively less active, i.e. their motion reduces. Similarly, following medical treatment, including surgery, a patients mobility may be effected, and may then change with time, for example increasing if the patient recuperates successfully, or reducing if the recuperation is not progressing satisfactorily, such as is the case if complications are encountered.

In many situations, there is a motivation to discharge the patients from hospital as soon as possible after treatment, to free up hospital places and enable the patient to recover at home. Also, in many of these situations it is the practice to arrange for the recuperating patient to receive visits from medical practitioners such as doctors or nurses at predetermined intervals in order to check that recuperation is progressing in a satisfactory manner and perhaps provide further care of treatment. Clearly, in some of these cases a patient may have being recovering so satisfactorily that the prearranged visit is not in fact necessary, and thus represents a waste of resources. On the other hand, it may be the ease that it would be desirable to visit the patient sooner rather than the time of the prearranged visit. In many circumstances it is desirable to provide medical intervention for a patient at a relatively early stage, before their activity has dropped to such a level that treatment becomes more difficult, complicated and/or expensive. In general terms, if a patients activity is declining because of the progression of a disease or because of some other factor, then it is preferable to intervene at an earlier stage, because the situation is then typically medically easier and requires fewer resources to remedy.

GB2334127A discloses an alertness monitor incorporating a movement sensor for generating signals dependant on the movements of the user. The signals can be used to generate an alarm signal if the wearer's movement does not exceed a predetermined threshold during a predetermined time interval. These alarm signals can be used to rouse the wearer, for example in the case where the wearer is a pilot of an aeroplane, or indeed to alert another person, for example where the wearer is a patient in a hospital or nursing home who has suffered a fall. Although the disclosed monitor has some use, it is a relatively unsophisticated means for monitoring motion, and would have limited value in the monitoring of recuperation of an orthopaedic patient post surgery for example.

### SUMMARY OF THE INVENTION

The present invention aims to provide a patient monitoring method or system which obviates or mitigates at least one of the problems associated with the prior art. Certain embodiments of the invention aim to provide a patient monitoring method and system that is a useful clinical tool in the monitoring of patient's, and in particular, although not exclusively orthopaedic patient's, both pre-treatment and post treatment.

According to a first aspect of the present invention there is provided a patient monitoring method comprising:
monitoring motion of a subject patient and generating motion data indicative of at least one aspect of the subject's motion;
processing said motion data to generate at least one statistical parameter of said data;
identifying a person type of the subject according to a plurality of characteristics;
accessing a database containing a plurality of stored, predetermined statistical parameters classified according to a plurality of said person types, each stored statistical parameter being a statistical parameter of motion data corresponding to a respective one of said plurality of person types and having been determined by a method comprising processing of motion data obtained by monitoring motion of at least one person having said respective one of said plurality of person types; and
processing at least one said generated statistical parameter and at least one of said stored statistical parameters corresponding to the person type of said subject patient.

Typically, the processing steps will be computer-implemented, that is carried out using a suitably programmed microprocessor or processors. The method provides that by processing both the actual generated statistical parameters of the subject motion data together with at least one stored statistical parameter of a person of the same type as the subject patient a wide variety of clinically useful results may be obtained. By processing (e.g. comparing) actual statistical parameters with “expected” statistical parameters of that person type (i.e. for a person whose circumstances correspond closely to that of the particular monitored subject) sophisticated decisions may he taken by the system, for example deciding whether or not to intervene.

Thus, in certain embodiments, the processing of the at least one generated statistical parameter and at least one of the corresponding stored statistical parameters comprises using at least one of the stored statistical parameters to analyse the at least one generated statistical parameter. This analysis may, for example, include a comparison, or for example an observation in the change or trend of a particular statistical parameter with time, and a comparison of that change or trend with the expected trend from the relevant stored data (i.e. from the expected statistical parameters stored in a persona profile corresponding to that patient type).

In certain embodiments of the invention, the method further comprises the step of determining a course of action for at least one of the subject and a medical practitioner according to the processed statistical parameters. This determining step may be carried out as part of the step of processing of the at least one said generated statistical parameter and at least one of said stored statistical parameters, and so the determining step may also be automatically performed by micro processing means suitably programmed (i.e. carrying out processing according to a pre-determined algorithm).

Thus, in certain embodiments said processing at least one said generated statistical parameter and at least one of said stored statistical parameters corresponding to the person type of said subject patient comprises processing at least one said generated statistical parameter and at least one of said stored statistical parameters corresponding to the person type of said subject patient to determine a course of action for at least one of the subject and a medical practitioner.

In certain embodiments the monitoring of motion of a subject patient comprises providing the subject with motion monitoring means to be worn or carried by the subject, the motion monitoring means being adapted to generate said motion data when worn or carried by the subject in response to motion of the subject.

In certain embodiments the motion monitoring means is adapted to detect steps taken by the subject and to generate motion data in response to each detected step.

Thus, the monitoring means may comprise a pedometer, a pedometer sensor, or a sensor based on and using pedometer technology. In certain embodiments, the monitoring means may be adapted to record each step taken, together with a respective time. The monitoring means may be worn by the subject patient at any suitable location on their person. The monitoring means may for example be secured to the subject patient at the ankle, knee, thigh or waist. The location in order of preference is ankle, waist, thigh and knee.

In certain embodiments the motion monitoring means further comprises processing means for processing said motion data, and the step of processing said motion data comprises processing motion data with the processing means of the motion monitoring means.

In certain embodiments the method further comprises the steps of transmitting motion data or processed motion data from the motion monitoring means and receiving the transmitted data at a remote location.

The transmitted signal containing the motion data or processed motion data in certain examples is a wireless signal. In alternative embodiments, the signal may be transmitted along a wire or cable, and in certain embodiments the monitoring means may be adapted to provide a combination of wireless and wire communication (for example including a GSM modem and antenna along with a suitable connector such as a USB port) for hard-wiring to some other component for downloading of the collected data.

In certain embodiments the method further comprises the steps of transmitting identification data indicative of an identity of the subject and receiving the transmitted identification data at the remote location.

In certain embodiments it is envisaged that the method further comprises the monitoring in addition to motion of one or more additional subject related measurements and generating subject related measurement data indicative of one or more further aspects of the subject patient's condition. This additional subject related measurement data may without further processing be used in combination with the resultant output of the processing of motion data in accordance with the method of the present invention.

Optionally the one or more additional subject related measurement data may be processed independently or in combination with the motion data (hereinafter the combination) using the same processing steps. Thus the one or more additional subject related measurement data or the combination may be processed to generate at least one statistical parameter of said one or more additional subject related measurement or the combination and this may be processed in a similar way to the motion data. This includes identifying a person type of the subject according to a plurality of characteristics; accessing a database containing a plurality of stored, predetermined statistical parameters classified according to a plurality of said person types, each stored statistical parameter being a statistical parameter of the one or more additional subject related measurement data or the combination corresponding to a respective one of said plurality of person types and having been determined by a method comprising processing of the one or more additional subject related measurement data or the combination data obtained by monitoring the one or more additional subject related measurements or the combination, of at least one person having said respective one of said plurality of person types; and processing at least one said generated statistical parameter and at least one of said stored statistical parameters corresponding to the person type of said subject patient.

It is envisaged that the one or more additional subject related measurements may be of any suitable physiological measurement, which may be determined remotely. Examples of such measurements include: temperature, heart rate, blood, chemical markers, resistance/current to estimate fat, oxygen saturation etc.

In a further embodiment the one or more additional subject related measurements may actually be patient initiated and/or defined. For example through the use of for example a keypad, voice monitor or data via a mobile phone e.g. text message, the subject patient may provide indications of existence and/or levels of pain, happiness, mental state etc. The patient may provide additional information gathered through self-administered tests/assessments such as blood glucose monitoring data, weight data etc.

In certain embodiments the motion monitoring means comprises a mobile telephone, and the step of transmitting comprises transmitting data from said mobile telephone.

Although the motion monitoring means may thus comprise a mobile telephone, in other embodiments the motion monitoring means may be separate from a phone but connectable to it. Thus, the user may carry or wear the motion monitoring means and then connected to a mobile telephone in order to transmit the signal containing the collected and/or processed data. Also, it would be appreciated in certain embodiments, the motion sensor of the monitoring means may be incorporated in a mobile telephone itself so that the user does not have to carry a separate phone and monitoring unit, instead the user just needs to carry the mobile telephone.

In certain embodiments the generated at least one statistical parameter comprises at least one statistical parameter indicative of a respective one of the following, each associated with an aspect of the subject's motion over a predetermined time interval: a mean value; a maximum value; a minimum value; a standard deviation; and a mode value.

In certain embodiments the motion data comprises motion data indicative of times at which the subject takes a step.

In certain embodiments the generated at least one statistical parameter comprises at least one statistical parameter indicative of a respective one of the following: a number of steps in a predetermined time interval; a mean number of steps in a predetermined time interval; a maximum number of steps in a predetermined time interval; a minimum number of steps in a predetermined time interval; a standard deviation of a number of steps in a predetermined time interval; a mode value of a number of steps in a predetermined time interval; a step symmetry; a mean step symmetry in a predetermined time interval; a maximum value of step symmetry in a predetermined time interval; a minimum value of step symmetry in a predetermined time interval; a standard deviation of step symmetry value in a predetermined time interval; a mode value of step symmetry in a predetermined time interval; a respective proportion of a predetermined time interval in which a step activity of the subject falls into at least one predetermined category; a mean step interval in a predetermined time interval; a maximum step interval in a predetermined time interval; a minimum step interval in a predetermined time interval; a standard deviation of step interval in a predetermined time interval; and a mode value of step interval in a predetermined time interval.

It will be appreciated that the above list of statistical parameters is not exhaustive, and in alternative embodiments other statistical parameters may be generated and indeed used in the analysis of the generated parameters. Also, in certain embodiments the at least one statistical parameter may be any one or any combination of a plurality of a listed parameters.

In certain embodiments the plurality of characteristics comprises at least two of: sex; age; body mass index; perceived activity level type; and surgical history.

Again, it will be appreciated that this list of characteristics is not exhaustive, and additional and/or alternative characteristics may be used in other embodiments of the invention. Also, the plurality of the characteristics used in embodiments of the invention may comprise any combination of two or more of the listed characteristics.

In certain embodiments the method further comprises providing a characteristics database containing data indicative of said subject's identity and data indicative of said plurality of characteristics of the subject, and said identifying a person type of the subject comprises accessing said characteristics database.

In certain embodiments the step of processing at least one said generated statistical parameter and at least one of said stored statistical parameters comprises comparing at least one generated statistical parameter with a corresponding stored statistical parameter.

In certain embodiments the step of processing at least one said generated statistical parameter and at least one of said stored statistical parameters comprises using a computer-implemented algorithm.

In certain embodiments the step of processing at least one said generated statistical parameter and at least one of said stored statistical parameters comprises determining a cost associated with the subject patient according to the generated at least one statistical parameter, determining an expected cost associated with the person type of said subject according to the corresponding stored statistical parameter or parameters, determining a cost associated with an action, and determining a course of action according to said costs.

In certain embodiments the course of action comprises at least one action for the subject.

In certain embodiments said at least one action for the subject comprises a change in motion activity.

In certain embodiments the method further comprises providing a signal to the subject, recommending said at least one action for the subject.

In certain embodiments the step of providing a signal to the subject comprises transmitting a signal.

In certain embodiments the step of providing the signal to the subject comprises making the signal available for access by the subject.

In certain embodiments the course of action comprises at least one action for a medical practitioner.

In certain embodiments said at least one action for the medical practitioner comprises surgery to be performed on the subject.

In certain embodiments the at least one action for the medical practitioner comprises visiting the subject.

In certain embodiments the at least one action for the medical practitioner comprises admission of the subject to a medical facility.

In certain embodiments the at least one action for the medical practitioner comprises discharging the subject from a medical facility.

In certain embodiments the method further comprises providing a signal to the medical practitioner, recommending said at least one action for the medical practitioner.

In certain embodiments the step of providing a signal to the medical practitioner comprises transmitting the signal to the practitioner.

In certain embodiments the step of providing the signal to the medical practitioner comprises making the signal available for access by the medical practitioner.

In certain embodiments the method further comprises using at least one of the generated motion data and the generated at least one statistical parameter of the subject patient to update said database by modifying at least one stored statistical parameter corresponding to the person type of the subject.

In certain embodiments the method further comprises populating said database with the plurality of stored, predetermined statistical parameters using a method comprising: for each person type, identifying at least one person having that person type, monitoring motion of the identified at least one person and generating motion data indicative of at least one aspect of the motion of the at least one identified person, processing the motion data from the at least one identified person to generate at least one statistical parameter of that data, and storing the generated at least one statistical parameter of the motion data of the at least one identified person in the database, classified according to person type of the at least one identified person.

In certain embodiments the step of identifying at least one person for at least one of the plurality of said person types comprises identifying a plurality of persons having that person type.

Another aspect of the invention provides a patient monitoring system (i.e. apparatus) comprising:
motion monitoring means adapted to monitor motion of a subject patient and generate motion data indicative of at least one aspect of the subject's motion;
processing means arranged to process said motion data to generate at least one statistical parameter of said data;
identification means arranged to identify a person type of the subject according to a plurality of characteristics;
a database containing a plurality of stored, predetermined statistical parameters classified according to a plurality of said person types, each stored statistical parameter being an expected statistical parameter of motion data corresponding to a respective one of said plurality of person types; and
processing means arranged to process at least one said generated statistical parameter and at least one of said stored statistical parameters corresponding to the person type of said subject patient.

In certain embodiments the processing means is arranged to process at least one said generated statistical parameter and at least one of said stored statistical parameters corresponding to the person type of said subject patient is further arranged to determine a course of action for at least one of the subject and a medical practitioner according to the processed statistical parameters.

In certain embodiments said motion monitoring means is adapted to be worn or carried by the subject, and is adapted to generate said motion data, when worn or carried by the subject, in response to motion of the subject.

For example, the motion monitoring means may be a unit provided with a strap or belt for attaching around the portion of the subject body.

In certain embodiments the motion monitoring means is adapted to detect steps taken by the subject and to generate motion data in response to each detected step.

In certain embodiments the motion monitoring means further comprises processing means adapted to process said motion data.

In certain embodiments the motion monitoring means further comprises transmitting means adapted to transmit motion data or processed motion data for reception at a remote location and the system further comprises receiving means arranged to receive the transmitted data at the remote location.

In certain embodiments the motion monitoring means is further adapted to store identification data indicative of an identity of the subject, and the transmitting means is adapted to transmit said identification data for reception at the remote location.

In certain embodiments the patient monitoring system further comprises means to determine the one or more additional subject related measurements. These may take the form of suitable sensors or monitors for determining the required measurement e.g. temperature may be monitored using a thermometer. Additional relevant processing means is also envisaged, which is consistent with the method of processing the one or more additional subject related measurements as discussed above.

In certain embodiments the patient monitoring system further comprises means to enable the patient to initiate and/or define the one or more additional subject related measurements. Examples of suitable means are discussed above in relation to the method of the present invention.

In certain embodiments the transmitting means is adapted to transmit data in a wireless signal.

For example, in certain embodiments the transmitting means adapted to transmit the data in a mobile telephone signal format for reception by a mobile telephone network.

In certain embodiments the generated at least one statistical parameter comprises at least one statistical parameter indicative of a respective one of the following, each associated with an aspect of the subject's motion over a predetermined time interval: a mean value; a maximum value; a minimum value; a standard deviation; and a mode value.

In certain embodiments said motion data comprises motion data indicative of times at which the subject takes a step.

In certain embodiments the system further comprises a characteristics database containing data indicative of said subject's identity and data indicative of said plurality of characteristics of the subject.

In certain embodiments the system further comprises means for inputting identity data and characteristics data into the characteristics database.

In certain embodiments said plurality of characteristics comprises at least two of: sex; age; body mass index; perceived activity level type; and surgical history.

In certain embodiments the processing means arranged to process at least one said generated statistical parameter and at least one of said stored statistical parameters is adapted to compare at least one generated statistical parameter with a corresponding stored statistical parameter.

In certain embodiments the processing means arranged to process at least one said generated statistical parameter and at least one of said stored statistical parameters is arranged to process said parameters using a predetermined algorithm.

In certain embodiments the processing means arranged to process at least one said generated statistical parameter and at least one of said stored statistical parameters is arranged to determine a course of action for at least one of the subject and a medical practitioner according to the processed statistical parameters, and is arranged to determine a cost associated with the subject patient according to the generated at least one statistical parameter, determine an expected cost associated with the person type of said subject according to the corresponding stored statistical parameter or parameters, determine a cost associated with an action, and determine a course of action according to said costs.

In certain embodiments the processing means arranged to process at least one said generated statistical parameter and at least one of said stored statistical parameters is arranged to determine a course of action for at least one of the subject and a medical practitioner according to the processed statistical parameters, and the course of action comprises at least one action for the subject.

In certain embodiments the at least one action for the subject comprises a change in motion activity.

In certain embodiments the system further comprises signalling means adapted to provide a signal to the subject, the signal recommending said at least one action for the subject.

In certain embodiments the processing means arranged to process at least one said generated statistical parameter and at least one of said stored statistical parameters is arranged to determine a course of action for at least one of the subject and a medical practitioner according to the processed statistical parameters, and the course of action comprises at least one action for a medical practitioner.

In certain embodiments the system further comprises signalling means adapted to provide a signal to a medical practitioner, recommending said at least one action for the medical practitioner.

In certain embodiments the system further comprises processing means adapted to update said database by modifying at least one stored statistical parameter corresponding to the person type of the subject using at least one of the generated motion data and the generated at least one statistical parameter.

### BRIEF DESCRIPTION OF THE DRAWINGS

Embodiments of the invention will now be described with reference to the accompanying drawings, of which:

FIG. 1 is a schematic representation of a patient monitoring system embodying the invention;

FIG. 2 is a schematic representation of part of another patient monitoring system embodying the invention;

FIG. 3 is a schematic representation of certain components of a patient monitoring system and method embodying the invention;

FIG. 4 is a patient activity profile plotting patient activity against time;

FIG. 5 is a schematic representation of components of a motion-monitoring device for use in methods and systems embodying the invention;

FIG. 6 is another patient activity profile illustrating the effects of an intervention on patient activity against time;

FIG. 7 is a plot of an activity variable against a cost variable for three different general patient activity types illustrating the intervention of the line of mobility and the line of immobility for a particular patient;

FIG. 8 is a flow chart illustrating the generation of different persona types according to a plurality of characteristics and the generation of associated persona profiles;

FIG. 9 is a flow chart illustrating the generation of a persona profile for a particular person type;

FIG. 10 is a flow chart illustrating the derivation of a profile of a single person of a particular person type;

FIG. 11 is a flow chart illustrating the processing of data in embodiments of the invention, including identification of a patient type, and processing of an actual activity profile of a subject patient together with an expected activity profile of a person of that type;

FIG. 12 is a flowchart illustrating the processing of motion data for a particular subject patient to generate an actual patient profile containing statistical parameters of the motion data; and

FIGS. 13-16 are plots of step data and statistical parameters of step data as a function of time for a particular observed subject.

### DETAILED DESCRIPTION OF EMBODIMENTS OF THE INVENTION

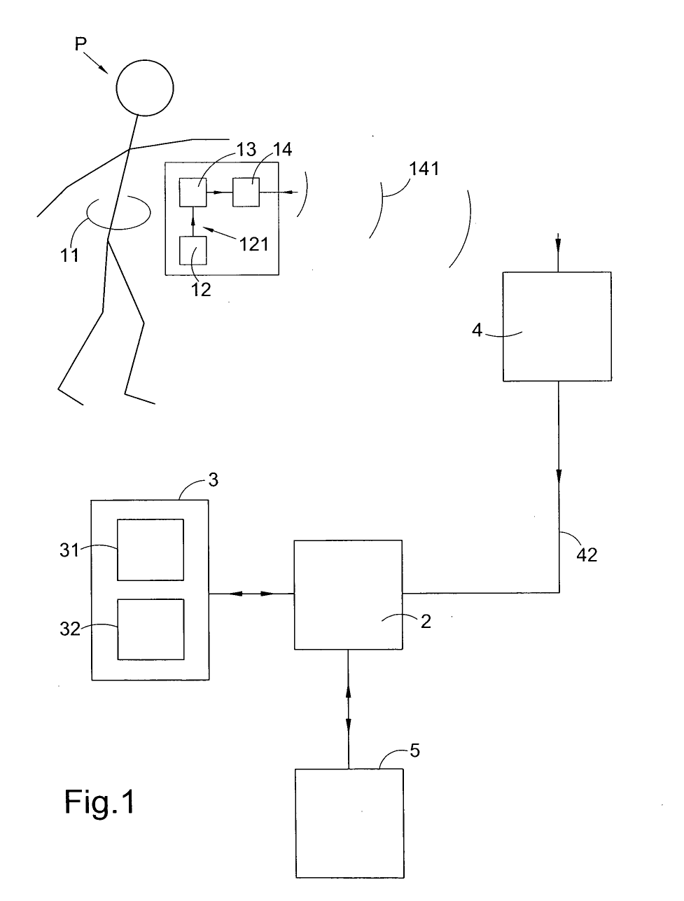

Referring now to FIG. 1, this illustrates the highly schematic form of a patient monitoring system (apparatus) embodying the present invention. The apparatus comprises motion-monitoring means in the form of a single monitoring device 1 adapted to be worn by a subject patient P. For this purpose, the device 1 is provided with an attachment means 11 in the form of a belt, but it will be appreciated that in alternative embodiments different attachment means may be utilised. The motion-monitoring device 1 includes sensing means in the form of a sensor 12 which is able to detect when the subject takes a step. In response to a detected step the sensor 12 generates motion data 121 which is stored in a memory 13. In this first embodiment the stored movement data simply comprises a time for each detected step, i.e. in use it fills up with a series of timed stamps, each corresponding to a particular step taken by the observed subject patient P. The monitoring device 1 also comprises a transmitter 14 arranged to transmit a wireless signal 141 containing the stored movement data 121 and additional data identifying the particular subject patient P. The monitoring device 1 in certain examples is arranged to transmit these data signals 141 continuously. In other embodiments, these signals 141 may be transmitted only at certain times, for example at regular, predetermined time intervals, in response to demand or prompt signals received from an external source, or when the memory has been filled to a predetermined level. The system further comprises receiving means 4 arranged to receive the transmitted signals 141 and to communicate the received signals to processing means 2. Although this processing means 2 is shown in FIG. 1 as a single unit, in practice it may be provided by a single processing device or a plurality of processes arranged in suitable communication with each other. In this first embodiment the processing means 2 is arranged to process the received motion data and generate at least one statistical parameter of that data. For example, it may generate a statistical parameter corresponding to the average number of steps detected by the sensor 12 in a predetermined time interval (e.g. an hour or day). Additionally, or alternatively, it may generate a statistical parameter corresponding to a maximum or peak step rate in a particular time interval and/or the portion of a predetermined time interval over which the step activity of the monitored subject P falls into a particular category (e.g. high, medium, or low). In this first example, all of the processing of the motion data 121 is performed by the processing means 2 (e.g. a microprocessor) which is remote from the observed subject P; no processing of the data 121 is performed by the monitoring device 1. However, in alternative embodiments, as described below, the monitoring device itself may perform processing on the motion data 121 before transmitting that processed data to the rest of the monitoring system. Also, it will be appreciated that although in this example the data from the motion sensor 1 is sent via a wireless signal, in alternative embodiments the motion data or processed motion data may be conveyed to the rest of the monitoring system via alternative means, such as by a plug-in connection to a suitable device and transmission over the interne. In such examples, the observed subject P may be required to connect (i.e. plug in) the monitoring device 1 to some communication port periodically (e.g. at a particular time each day).

Returning to the present embodiment, the processing means 2 is also arranged to identify the person type of the observed subject P according to a plurality of characteristics. The processing means 2 does this by using the identification data received from the motion sensor 1 and accessing a database 3 containing data 32 on the relevant plurality of characteristics for that particular subject. For example, the database may contain data for the particular subject identity corresponding to that subject age, sex, weight, body mass index, medical history including surgical history, and possibly other factors. In the present example the micro processing means 2 accesses the database 3 and uses that characteristics data 32 to determine (using a suitable algorithm) a particular person type or category into which the observed subject P falls. In alternative embodiments, the person type of the particular subject may already have been determined and the processing means 2 may simply then consult an appropriate database to learn the person type stored for the particular subject's identity.

The database 3 also comprises a plurality of stored persona profiles 31, which can also be regarded as examples, templates, or targets. These persona profiles are classified according to a plurality of different person types, with each persona profile corresponding to one of those types. Each persona profile contains at least one statistical parameter that is an expected statistical parameter of motion data corresponding to a person of a particular type. In other words, the statistical parameters of motion data stored in the persona profile database are examples of the statistical parameters that one might expect to obtain from observation of she motion of a person of that type. Preferably, these persona profiles have been generated by observation of actual people of the respective types, using appropriate analysis of motion data obtained from those observations. Preferably, each persona profile contains statistical parameters that have been obtained by analysis of motion data and a plurality of people of that particular type.

The processing means 2, having identified the person type of the observed subject P is arranged to process at least one of the generated statistical parameters of the observed subject's motion data and at least one of the stored statistical parameters of the persona profile corresponding to the person type of the subject patient P and to determine a course of action (i.e. decide on something to be done) according to the results of that processing of the generated and stored statistical parameters. In other words, the processing means 2 accesses the persona profile database and uses the stored, expected statistical parameters to analyse the actual statistical parameters obtained from analysis of the subject's motion to make a decision as to what to do. The determined course of action may comprise just a single action, which may he something for the patient P to do or something for a Medical Practitioner to do, or indeed may comprise a plurality of actions. in certain embodiments, the processing means may be adapted to send a signal back to the observed subject P recommending one or more actions. However, in the embodiment shown in FIG. 1, the processing means 2 is simply arranged to provide information on the determined course of action to a medical practitioner via a suitable interface or terminal 5. Thus the processing means 2 is able to send a signal to the medical practitioner, which may for example be a recommendation to visit the subject, admit the subject to a medical facility, discharge the subject from a medical facility (if appropriate) or perform some treatment on the subject, such as surgery. In the present example, the medical practitioner via the terminal or interface 5 is also able to provide data to the processing means 2 for use in updating the database 3. This data may, for example, comprise data identifying a particular subject, and characteristics data for that subject, for example including age, sex, perceived activity level, weight, body mass index, medical history (which may include surgical history) and indeed other information.

Thus, it will be appreciated that by identifying the particular person type of the observed subject P and then using predetermined statistical parameters of motion data for that particular person type to analyse the statistical parameters of the actual motion data, the micro processing means can be arranged to make sophisticated clinical decisions. It is not simply comparing motion data with a single predetermined, and perhaps arbitrary threshold; instead statistical parameters of observed motion are compared with expected values corresponding to the particular circumstances of the observed patient P.

For example, if the observed subject P has undergone knee replacement surgery the system maybe arranged to monitor motion of the subject at home, after discharge from the hospital in order to determine whether recovery is progressing satisfactory, and in deed whether a visit by a medical Practioner is required or if re-admission into a hospital is required in order to do this, the processing means 2 can look at a statistical parameter such as the number of steps being taken by the patient per day and see how this progresses on a daily basis. Then, rather than just simply comparing a particular daily step total with an arbitry fixed threshold, the processing means can compare the daily progression with the daily progression of a persona profile obtained by observation of previous patients who have undergone the same surgery. On a simple level, for example, the number of steps taken on a twentieth day following surgery could be compared with the typical number of steps taken on that day following surgery from the persona profile, and according to the result of the comparison a signal may be generated. This could be a signal to the patient to try to increase activity (i.e. number of steps they can take per day), a signal to a medical Practioner to visit the patient because the number of steps (i.e. the activity level) is not high enough, indicating the recover from the surgery is not progressing adequately, or indeed may be an alert signal or warning because the number of steps being taken is too large, therefore risking harming of the recovery process. Using a more sophisticated approach, rather than just using the statistical parameters for a particular day, a trend in those parameters may be compared with a typical trend from the stored persona profile.

Certain embodiments of the present invention provide patient monitoring systems and patient monitoring methods which automatically performs cynical analysis of patient activity data gathered by a motion sensor. In certain embodiments, the data from the motion sensor is analysed and then the analysed data is automatically transmitted to a central database. Systems embodying the invention can be particularly user-friendly, as they require the patient to do no more than wear the sensor and charge the battery periodically.

Certain embodiments of the invention are particularly directed to the monitoring of orthopaedic patients, and for such applications the monitor device is may be ranged to monitor movement either by use of a simple, electro-mechanical pedometer, or using more sophisticated accelerometer based measuring technology. Thus, it would be appreciated that the monitoring means employed in embodiments of the invention may include pedometer sensors which sense body motion and count footsteps. Such monitoring devices, incorporating pedometer technology, may be worn all day if desired, and are able to record a total number of steps taken. Various pedometers for pedometer technology may be incorporated in embodiments of the invention. For example, a pedometer may comprise piezo-electric accelerometers, coiled spring mechanisms; or hairspring mechanisms. The pedometers may use tuned pendulum technology, accelerometers, and/or electronics to count steps.

Although certain embodiments monitor patient motion by means of a pedometer or pedometer technology incorporated in a measuring device to be worn by the patient, it should be appreciated that in its broader sense the present invention is not limited to using such motion monitoring means, and other means for detecting motion and for generating motion data may be used in alternative embodiments. For example, the patient may wear a passive device, and the system may comprise means for attacking that passive device. Similarly, the motion monitoring means may comprise a GPS receiver and means for logging position of the subject against time. The downloaded positional data may then be used as an indication of patient motion.

However, and advantage provided by pedometer-based sensing systems is that they are able to provide a relatively simple and cost effective means of monitoring patient movement. Their ability to detect individual steps is particularly useful in the monitoring of orthopaedic patients, and indeed is able to provide information on useful features such as step symmetry. Thus, even a simple pedometer simply logging the time of each step can provide data which is clinically useful.

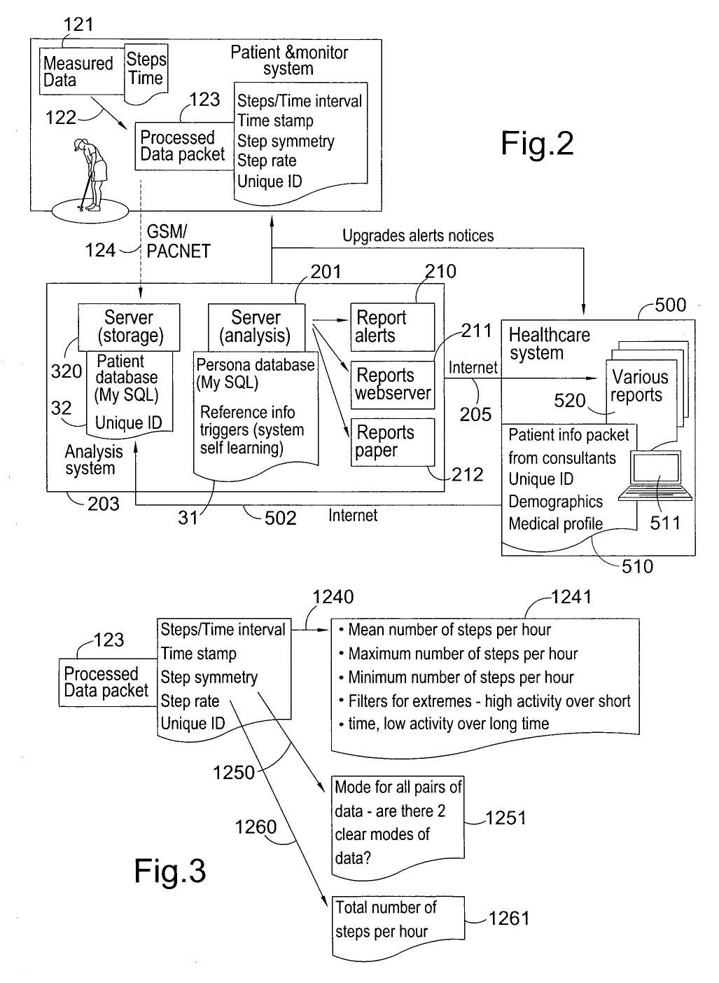

Referring now to FIG. 2, this shows part of a patient monitoring apparatus and system embodying the invention in which a pedometer-based monitoring unit has been worn by a subject patient and has generated measured data 121 comprising a respective time in which each step has been taken by the subject. In this embodiment, the monitoring device comprises processing means and has performed some initial processing 122 of the measured data 121 in order to generate process data package 123. This processed data package 123 contains data indicative of the number of steps taken in a particular time interval. It also comprises a time stamp (which may, for example, indicate the day and the time of day corresponding to the particular packet). The processed data packet 123 also provides data indicative of step cemetery (which can be determined by looking at the time intervals between successive steps), and data on step rate. Additionally, the processed data packet also contains unique identification data indicative of the identity monitored subject.

The motion monitoring means then transmits a wireless signal 124 (which may for example be in GSM/pacnet format) to an analysis system 203. This analysis system may be at a single location, or may for example be distributed over a number of locations. The analysis system 203 includes a first server 320 which performs a storage function that holds a database 32. This database 32 comprises a patient database e.g. in MySQL format, storing data on patients along with the patient's unique identification data. The analysis system 203 also comprises a second server 201 arranged to perform an analysis function. This analysis server 201 is arranged to access a persona data base (which again may be in MySQL format) containing the stored predetermined statistical parameters corresponding to different patient types that have already being determined and stored in suitable storage means. The analysis server 201 is also adapted to access reference information triggers which are triggers or parameters against which receive data or analysis results can be compared to trigger systems self learning, that is updating or modification of the data stored in the persona data base according to the actual motion data or statistical parameters generated from the actual motion data for a particular observed subject. Thus, as the system is used to monitor and analyse the motion of subjects, if you can take into account the subject types and use that information to update the stored persona profiles, progressively improving their clinical value as targets or models against which a patients motion can be compared. The analysis server 201 is adapted to perform further processing of the processed data received in the processed data packets from the monitoring means and generate various statistical parameters of that received data. The analysis server is also adapted to determine the identity of the monitored subject and to analyse the generated statistical parameters using the pre-determined stored (i.e. target) statistical parameters in the relevant persona database. According to the result of that analysis, the server 201 then determines (i.e. decides on) a recommended course of action. The determined course of action is then used to generate appropriate signals or messages which in this example comprise report alerts 210 for sending to the monitored subject and/or a medical practitioner, reports on a report web server 211 for access via the Internet 205 by a medical practitioner, and paper reports 212 for the monitored subject and/or medical practitioner. In FIG. 2 the monitoring system is shown to include part of a health care I.T system 500 this comprises a plurality of terminals 511 by means of which the medical practitioners can communicate with the analysis system by means of the Internet 205, 502. The medical practitioners are able to input data 510 to the analysis system via the Internet 502 and in particular to the patient database 32. The information provided by the medical practitioners may include, for example, patient information packets from Consultants, unique identification data for the particular subject, demographic information, and medical profiles or history, which may of course include surgical history of the patient. The Medical Practitioners are able to receive various reports 520 via the health care IT system 500. These reports may again be provided to the system 500 via the Internet 205 and in certain embodiments of the invention include recommended courses of actions to be taken in connection with a particular observed subject, for example to perform an intervention such as a surgical intervention or a visit or an admission to or discharge from a medical facility. The health care system 500 is also adapted to receive upgrades, alerts, and notices from the analysis system, and the analysis system 203 is also adapted to transmit upgrades, alerts and notices to the motion monitoring means carried by a patient which in this embodiment is of course adapted to receive and process such signals.

Referring now to FIG. 3, this is a schematic representation of processing of data from a processed data package 123 to generate various parameters including statistical parameters of the collective motion data. For example, the pre-processed data with information on the number of steps in a particular time interval may be processed by the analysis system 203 in a processing step 1240 to determine a number of statistical parameters 1241, including a mean number of steps per hour, a maximum number of steps per hour and a minimum number of steps per hour. The processing may also be arranged to filter the steps/time interval data for extremes for example to identify relatively high activity over a short time, and/or low activity over a long time. The step symmetry data may be processed by the analysis system 203 in a step 1250 to determine a statistical parameter 1251 which is a mode of all pairs of step data. The system is then arranged to look at that mode information to determine whether there are two clear modes of data, which would indicate day, step asymmetry. Lastly, the step rate information may be processed in a step 1260 to determine a total number of steps per hour 1261 which of course is another statistical parameter of the generated motion data.

It will be appreciated from the above description of FIGS. 2 and 3 that embodiments of the invention are able to offer clinical analysis of data collected on the motion of a particular patient and make the clinical analysis available for clinicians to access via the internet, or alternatively to proactively deliver the information to the clinicians. The processing system 203 is able to determinate course of action according to the results of this analysis and therefore provides a recommendation for action to be performed by the patient and/or clinician.

The health care system is constantly trying to decide where best to spend money on individual patients. In many instances, there is pressure to discharge patients from hospitals as early as possible after treatment (which may include surgery) to reduce hospital costs. However, in the past the cost to the community to which the discharged patients are returned is not understood or even not recognised.

The present inventors are aware that a patient activity level is related to the costs (less active is to typically more costly, and vita versa), whether that cost is direct or indirect, and measure if activity can easily be measured in embodiments of the invention using simple tools like pedometer. in embodiments of the invention, by profiling expectations from different patient groups it is possible to prioritise spend on individual patients by accessing their actual with expected profile (i.e. accessing statistical parameters of their actual motion compared with expected statistical parameters) and according to the result of that assessment decide on a course of action which gives both beneficial to the patient and cost effective. Resources, which of course are always finite, can thus be targeted where there will be of greatest benefit.

As will be appreciated of FIGS. 2 and 3, a patient monitoring system embodying the invention comprises a data base of normal and patient activity profiles, a means to measure patient activity, a means of transferring patient activity data to a processor for analysis, a means of reporting analysed information to various users, and a means of inputting patients medical data to the system which is relevant for the analysis.

The analysis system 203 of FIG. 2 is arranged to take patient activity profiles (that is profiles containing statistical parameters of motion data actually obtained from observation from a particular subject) and match those profiles with corresponding persona profiles (pre-determined, and stored on a data base). The standard persona profile for use of the analysis of particular patients data is selected from a database of standard persona profiles and matched to the particular patient type using the patient-specific demographics and medical profiles. In other words, the statistical characteristics of the patient are used to identify the relevant patient type and then the persona profile for that type is used in the analysis of obtained results. For the analysis, “normal” and “operated” profiles may be available for each persona group (type). For example, if an observed subject has not yet undergone a surgical procedure, their observed activity profile should be compared with the nominal “normal” persona profile for that person type (i.e. a persona profile corresponding to a statistical sample of people who have not undergone a relevant surgical procedure). Comparison of the actual profile with the normal persona profile can thus be used to determine whether or not it would be appropriate or cost effective to perform a surgical procedure on the subject patient. Alternatively, if the patient has undergone a surgical procedure, there may be little point in comparing their activity profile with that of a “normal” persona, this is the persona is of people who have not undergone the same procedure. Instead, in embodiments of the invention the actual profile of the person having undergone the surgical procedure is analysed using the persona corresponding to a sample of previous patients having undergone that procedure to give a more sophisticated and useful clinical analysis. For example, the system is able to decide whether the recuperation of a patient, post surgery, is progressing adequately based on an expected progression determined from observation of previous patients.

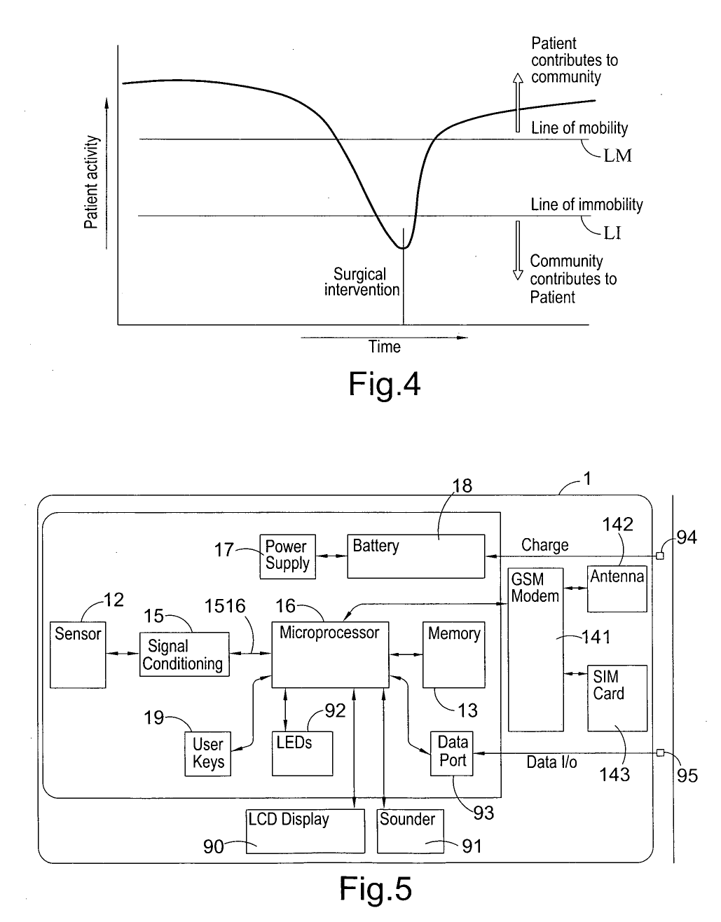

With regard to patient activity with time, a typical patient activity profile is shown in FIG. 4. Initially, the “patient”, i.e. a possible candidate for surgical intervention, has relatively high activity. With time, however, as the patients disease progresses, their activity level declines until such a time that they become relatively immobile and require intervention. The timing of a surgical intervention is indicated on the figure. After intervention a recovery path will be followed until an acceptable level of mobility is attained. On the figure there are shown two horizontal lines, a line of mobility LM above which the patient can be considered to make a positive contribution to the community, and a line of immobility LI below which the net contribution is from the community to the patient.

A patient monitoring system and apparatus embodying the invention can be used to monitor the activity of the patient whose typical profile is shown in FIG. 4. The activity monitoring device 1 of such a system is shown schematically in FIG. 5. This device comprises an accelerometer sensor 12 arranged to measure patient activity when the patient wears or carries the device 1. In response to patient activity this sensor 12 generates motion data 121 which is conditioned using signal conditioning means 15. The condition signal 1516 is then supplied to a microprocessor 16. The device 1 further includes a memory module 13 an LCD display 90, a plurality of LED's 92 a sounder 91 (i.e. sound generation means) and a global system for global communications (GSN) modem. This modem is connected to an antenna 142 and a sim card 143. The device also comprises a battery 18, power supply means 17 adapted to control the supply of power from the battery 18 to power the rest of the unit 1, a number of user keys 19 for inputting data and/or signals to the microprocessor, and a data port 93 connected to a data input/output terminal 95 for connection to other equipment (for example for direct downloading of collected, processed data from the sensor 12 rather than sending that data or processed data wirelessly via the GSM modem 141). There is also a charging terminal 94 via which the battery 18 can be charged from an external power supply. In this embodiment the microprocessor 16 is erased to perform minor analysis on the conditioned data signal 1516 and then that processed data can be communicated to a central database/analysis system via the GSM modem 141 and antenna 142. The memory 13 is able to store processed data generated in between transmissions. The memory is also able to store unique identification data relating to the particular subject carrying or wearing the device 1.

Thus, data transfer from the monitoring unit 1 shown in FIG. 5 can be by mobile phone network, by local wireless communication such as blue tooth, or by physical connection to a network using a dock or USB connection. Thus, in FIG. 5 the connection 95 may be a USB connection. The GSM modem and antenna 141, 142 of the device in FIG. 5 are able to transmit data to an analysis system via a GSM based network with a user data payload of at 160 bytes. Data can thus be communicated using standard text messaging.

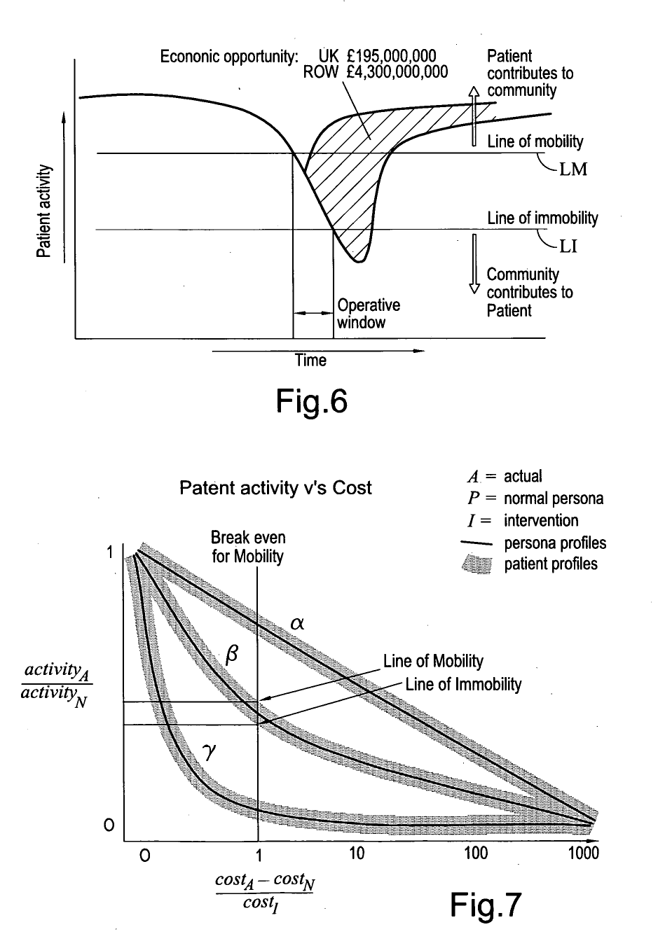

As will be appreciated from the description of FIGS. 2 and 3, the method of determining a course of action (i.e. concluding an action or actions to be carried out, for example to operate, to keep in hospital, to keep at home) utilised in embodiments of the invention is dependant on the analysis of the data (i.e. analysis of the motion data from the observed patient). In certain embodiments, the analysis conducted generates the key variables of line of mobility and line of immobility (discussed briefly above), the area between which may be called the operative window. This is shown on FIG. 6, and it would be appreciated that patient activity should be monitored and intervention performed in this window, i.e. before the patient activity dropped at such a level that they represent a net cost to the community. The line of mobility is defined by that activity level above which a patient's net cost is a positive value to the community (taking into account factors such as patient attitude and well being, salary, charity, family support, nursing visits, doctors visits, drugs, and special equipment). The line of immobility is defined by that activity level below which a patient's net cost is a negative value to the community. The intersection of these lines with the patient's activity profile defines the operative window, which will of course vary according to particular patient, age, nature of the disease etc.

In certain embodiments, in determining a course of action for a patient who has not yet undergone a particular treatment (e.g. surgery) a “normal” persona is selected from the pre-prepared database corresponding to the expected patient population. That is, the persona selected to analyse the motion of the data corresponds to expected normal activity of people having the same general type as the observed patient. An activity variable is calculated as activity=activity subscript A/activity subscript M where activity subscript A represents a statistical parameter of movement data actual measured on the patient, and activity subscript M is the corresponding statistical parameter from the normal, un-operative persona profile, i.e. the statistical parameter of the motion data that one would expect to obtain. For example, activity subscript A may be the actual total number of steps taking by the subject patient on a particular day, and activity subscript N may be the expected total number of steps to be taken in a day by a persona of the particular type.

In determining the course of action, embodiments of the invention are also able to generate a cost variable according to the equation cost=(cost subscript A−cost subscript N)/cost subscript I where cost subscript A is an actual cost associated with the monitored subject (whose not yet undergone surgical procedure) and cost subscript N is a cost that one would normally expect to be associated with an un-operated person of that type. Cost subscript I is a cost associated with an intervention. Thus, generally speaking, embodiments are of the invention are able to determinate cost variable in terms of the overall cost of a person in their present condition to the community relative to the cost of an intervention, which could in theory return the patient to a condition in which they make a net positive contribution to the community.

FIG. 7 shows the activity and cost variables plotted for three different patient types. The line a corresponds to a low mobility patient with significant costs relating to their immobility (e.g. due to weight, age, or other associated disease factors). γ represents a highly mobile patient with low associated costs. β represents a patient of medium mobility with medium associated costs. Tolerances having a pre-determined width on the activity access are defined around the solid lines indicating the plot of activity variable verses cost variable to accommodate subtle differences within the persona matches. The break even point is for a cost variable value of 1 shown intersectionally profile lines vertically. The inter section of this line with the upper and lower extremes of the patient profile band (i.e. the tolerance band on either side of the solid profile line) generates the activity variable associated with the line of mobility and line of immobility respectively. From FIG. 7 it will be seen that for a low mobility patient with already significant cost relating to their mobility, (line α) their actual activity only has to fall a small amount compared with the expected activity of a person of that type to reach the break even point for mobility, and hence make intervention desirable from a cost effectiveness point of view. Conversely, for a highly mobile patient (see plot γ) their activity must drop very significantly compared with the expected activity for a person of that type before it becomes cost effective to intervene.

It will be appreciated that according to the particular medical history of a patient, different algorithms may be used to determine a recommended course of action. These algorithms may take into account costs associated with various circumstances and procedures. The above example of plotting patient activity verses cost to determine whether or not to intervene was based on the cost of a surgical intervention to restore the activity level of the patient. In other examples of the patient monitoring system embodying the invention being used, the monitored subject may already have undergone a surgical procedure, and then their motion data may be analysed using expected statistical parameters of other people undergone that procedure to determine whether or not a visit is required, for example. On a relatively simple level, this analysis may result in recommending a visit to a discharged patient post surgery at an earlier time then would otherwise being the case, based on their analysed motion, and thereby intervening quickly to prevent a problem worsening. Conversely, the course of action determined for another patient may be a decision not to visit the patient because their activity profile, when analysed using the expected profile, is perfectly satisfactory. This saves the cost of what would have being an unnecessary visit. Thus, by performing sophisticated statistical analysis of patient motion data in conjunction with expected profiles embodiments of the invention are able to provide better use of medical resources by targeting them where they are actually needed.

Returning to the system described with reference to FIG. 2, it will be appreciated that embodiments of the invention provide a data reporting function. They enable customisable reports to be made able to meet the needs of a patient, a general practitioner and a hospital for example. Data may be supplied in the reports for each or just some of these users. For example, data can be provided to the patient to encourage a prescribed activity level for better recovery of the patient post operatively. Thus, the monitoring system may send a message to the patient recommending an increase in activity level, if the system determines that the previous activity level was too low), alternatively it could recommend that activity levels be reduced if the observed levels were statistically too high such that they risked impairing the recovery process. In terms of delivering data to a general practitioner, the result of the analysis performed by embodiments of the invention enable the general practitioner to justify expenditure on particular clients and to spot any abnormalities after an operation at an early stage. In terms of providing data to hospital personnel and also surgeons, the results of the analysis performed by embodiments of the invention are able to support the selection of a particular procedure, provide guidance to the timing of an operation (i.e. when to intervene) and also provide a means of early detection of post operative complications or failures.

In embodiments of the invention, medical practitioners are able to supply data to the system. For example, data may be required from a medical practitioner for two purposes. Firstly, to synchronize the patient with a unique identification code or data, and secondly to provide patient data to enable the system to select the correct database persona profile for use in analysing the patients movement. Data can be provided to the practitioner through a web interface and may also be sent from the practitioner to the analysis system via the Internet. The data may then be stored for subsequent profile comparisons during the treatment and finally added in to the persona database at the end of treatment to supplement decision making and analysis process.

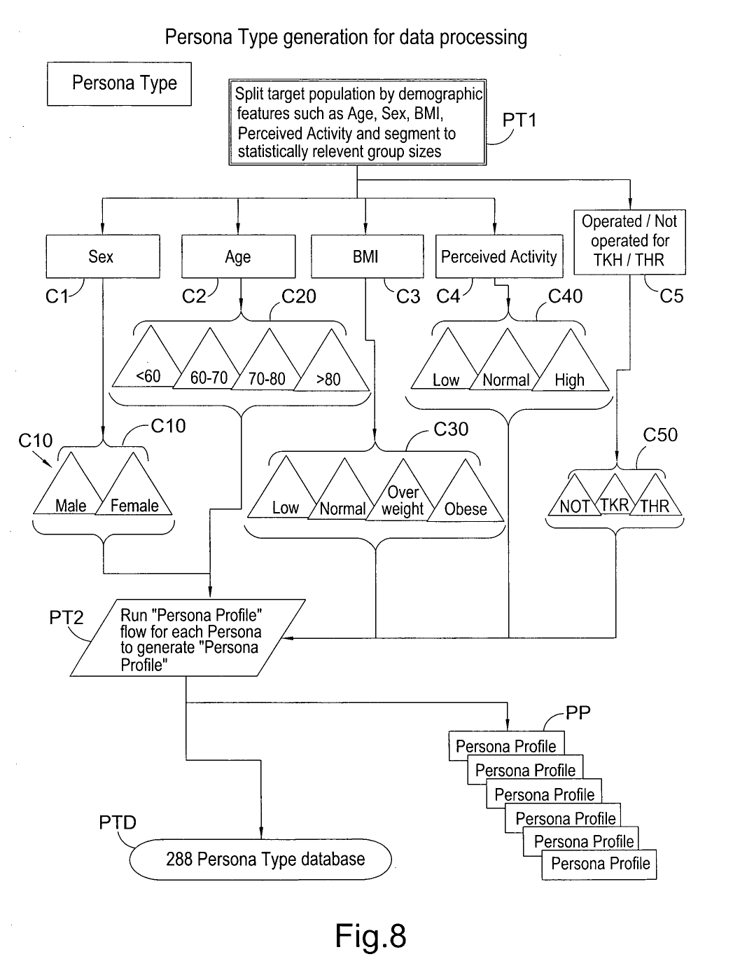

Referring now to FIG. 8, this is a flow chart of a method of generating persona types and corresponding persona profiles in certain embodiments of the invention. It will be appreciated that this method, and indeed other methods described throughout the specification and also processing operations described in this specification will typically be computer-implemented, that is carried out by suitably programmed microprocessing means. In step PT1 the target population is split by demographic features such as age, sex, body mass index (BMI), perceived activity, and segmented to statistically relevant group sizes. Thus, the characteristics used to define each person type (or persona type) are sex C1, age C2, body mass index C3, perceived activity C4, and a final characteristic C5 indicative of the surgical history of the person. In this particular example, this surgical history characteristic C5 is used to place the particular person in one of three corresponding categories C50, a “not operated” category for persons who have not undergone one of the particular surgical procedures, a TKR category of persons who have undergone total knee replacement surgery, and a THR category for patients who have undergone total hip replacement surgery. It will be appreciated that these categories are merely examples, and further categories or alternative categories may be used in alternative embodiments as appropriate.

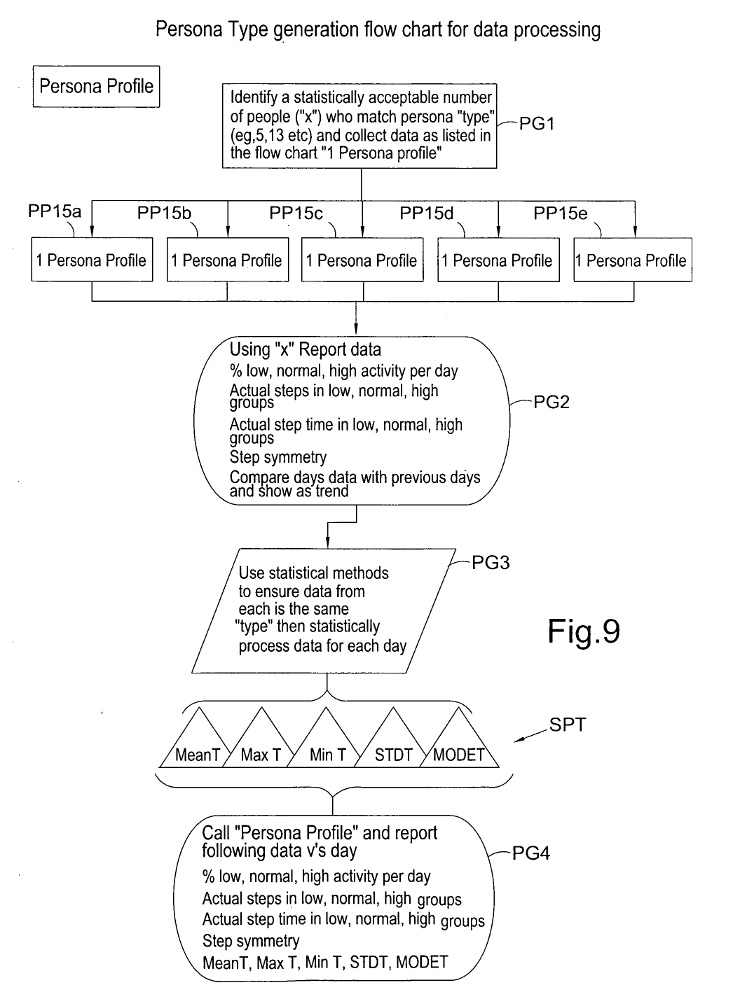

According to the sex characteristic C1, a person that falls into one of the two corresponding categories C10, i.e. male or female. In this example the age characteristic C2 is used to define one of four age categories C20 for the person, that is below 60, 60-70, 70-80 and over 80. Similarly, according to the body mass index characteristic C3, four categories are defined C30, being low, normal, overweight, and obese. The perceived activity category C4 is an indication of how generally active the person is perceived to be, and is categorised according to three categories C40, that is low, normal, and high. Thus, in this particular example the division of the various characteristics C1-C5 into categories C10, C20, C30, C40, C50 results in 288 different persona types, each persona type corresponding to a different combination of those categories. Instead PT2 the “persona profile” algorithm is run (as will be described below with reference to FIG. 9) for each one of the 288 different persona types, and in each case generating a persona profile PP corresponding to that type. These persona profiles PP may then be saved in an appropriate database, categorised according to persona type. The 288 persona types may also be stored in a persona type database instead PTD.

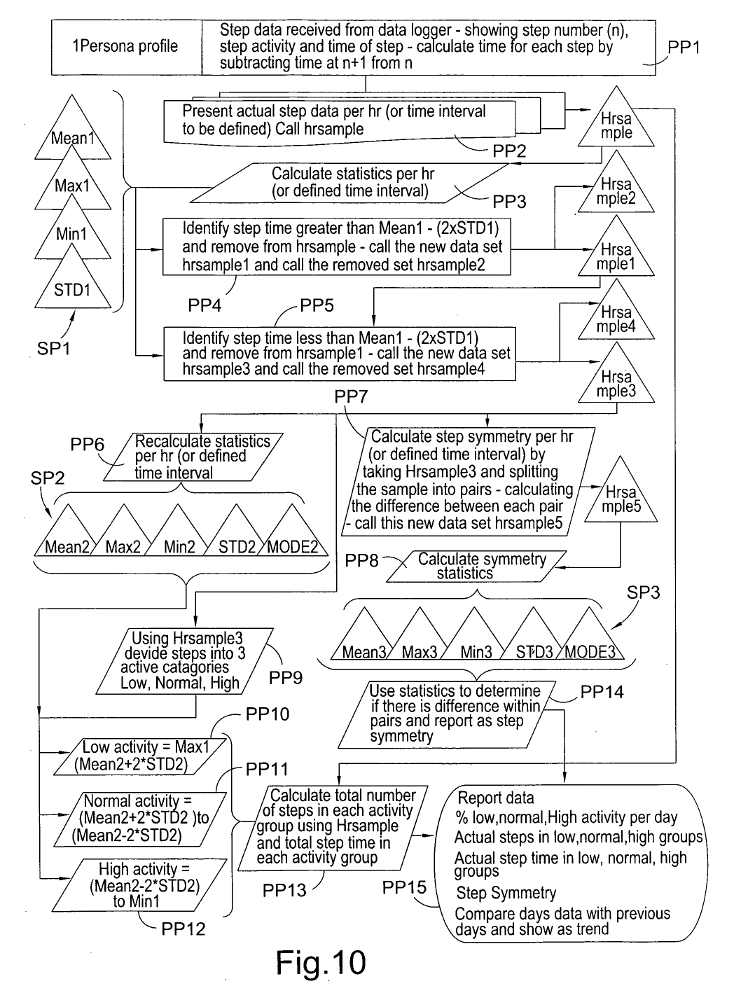

Referring now to FIG. 9, this is a flow chart illustrating the algorithm used to generate persona profiles in embodiments of the invention. In step PG1 the method identifies a statistically acceptable number of people (X) who match the particular persona type (for example five people, thirteen people, etc, and then motion data is collected and processed as described in the flow chart “one of persona profile” shown in FIG. 10. In the particular example shown in FIG. 9, five people have been identified matching the particular persona type and five individual persona profiles PP15A-E have been generated, one for each of these individuals. Each individual persona profile PP15 contains report data which contains statistical parameters of motion data actually observed on a particular subject. In this example each set of report data contains data indicative of the percentage of low, normal, and high activity by the patient per day, the actual number of steps in the low, normal, and high groups, the actual step time in low, normal, and high groups, data indicative of step symmetry, and also trend data showing the days data compared with the previous day's data. In step PG2 the X (i.e. in this example five) sets of report data are used. In step PG3 statistical methods are used to ensure that the data from each individual persona profile PP15 is the same type, and then the data for each type from each of the X individual persona profiles PP15 is statistically processed for each day. For example in this context one “type” is the percentage of high activity per day. Another type is the percentage of low activity per day. Another type may be the actual number of steps taken in a particular day. Another type may be the step symmetry for a particular day. Thus, in general the type in this context may be labelled as a variable T. Step PG3 thus processes the data of each type T from each of the X sets of the report data to generate statistical parameters of the data of that type T. These statistical parameters are labelled SPT in FIG. 9. For each data type T there are generated a mean value, mean T, a maximum value, max T, a minimum value, ruin T, a standard deviation of the five sets of data for that type, STDT, and the mode value of the type, mode T. Then, in step PG4 the resultant generated statistical data is labelled as a persona profile for the particular patient type, and contains a report of the following data versus day (i.e. as a function of day on a daily basis): the percentage of low, normal, and high activity per day, the actual steps in low, normal, and high groups, the actual step time in low, normal, and high groups, step symmetry, and the mean T, max T, min T, STDT and mode T statistical parameters for each of the various data types.

It will be appreciate from the above description that the various statistical parameters of motion data stored in the persona profiles are merely examples, and in alternative embodiments different statistical parameters maybe generated and saved, to suit the particular requirements of the monitoring system.

Referring now to FIG. 10 this is a flow chart illustrating the algorithms used in the embodiments of the invention to generate a single persona profile PP15 of a person of a particular person type for processing using the algorithms shown in FIG. 9 to generate an overall persona profile for that type (i.e. the profile taking into account motion data and statistics thereof for a plurality of people of that type). In the algorithm of FIG. 10, instead PP1 step data is received from a data logger (i.e. a pedometer or other such device providing information on steps taken by the monitored subject). The step data shows step number (N), step activity, and time of step, and in step PP1 a time for each step is calculated by subtracting the time for step N+1 from the time for step N. In step PP2, the actual step data per hour, or some other predetermined time interval to be defined is presented and called hrsample. In step PP3 statistical parameters per hour, (or some other defined time interval) are calculated from the data hrsample to generate statistical parameters SP1 including mean, maximum, minimum, and standard deviation values of step time. Step PP4 identifies data corresponding to a step time greater than the means step time value +2× the standard deviation of the step time value and removes those data entries from the set hrsample, and the new data set is called hrsample1 and the removed set of data is called hrsample2. Step PP5 identifies the data in hrsample1 corresponding to a step time less than the mean step time −2× the standard deviation of step time and removes that data from the set hrsample1, calling the new data set hrsample3 and the removed set hrsample4. Step PP6 operates on data set hrsample3 and recalculate statistical parameters of that data set per hour, or for some other defined time interval generating a set of statistical parameters SP2, including mean, maximum, minimum, standard deviation and mode values relating to the data of hrsample3. Step PP9 uses hrsample3 and divides the step data into three activity categories, nominally low, normal and high. Step PP10 determines the data from hrsample3 falling into the low activity category, that activity category being defined as that in which the step time falls in the range (max step time (from the statistical SP1)) to (mean step time from SP2+2× standard deviation of step time from SP2). Similarly, step PP11 determines the data from hrsample3 falling in the category of normal activity, that category being defined by the range (mean from SP2+2×STD from SP2) to (mean from SP2−2×STD from SP2). Similarly, step PP12 determines the amount of data from hrsample3 falling in the high activity category, defined that data falling in the range (mean from SP2−2×STD from SP2) to (min1 from SP1). In step PP13 the total number of steps in each activity group is calculated using hrsample and total step time in each activity group. In step PP7 step symmetry per hour (or other defined time interval) is calculated by taking hrsample3 and splitting the sample into pairs, calculating the difference within each pair, and calling this new data set hrsample5. Step PP8 calculates the symmetry statistics from hrsample5, generating a set of statistical parameters SP3 including mean, maximum, minimum, standard deviation and mode values. Step PP14 uses these statistical parameters SP3 to determine if there is any difference within pairs, and reports a result as step symmetry. Steps PP13 and PP14 yield report data PP15 which contains data indicative of the percentage in the low, normal and high activity categories per day, the actual steps in low, normal and high groups, the actual step time in low, normal and high groups, symmetry, and also data comparing the days data with the previous days data and displaying that data as a trend.

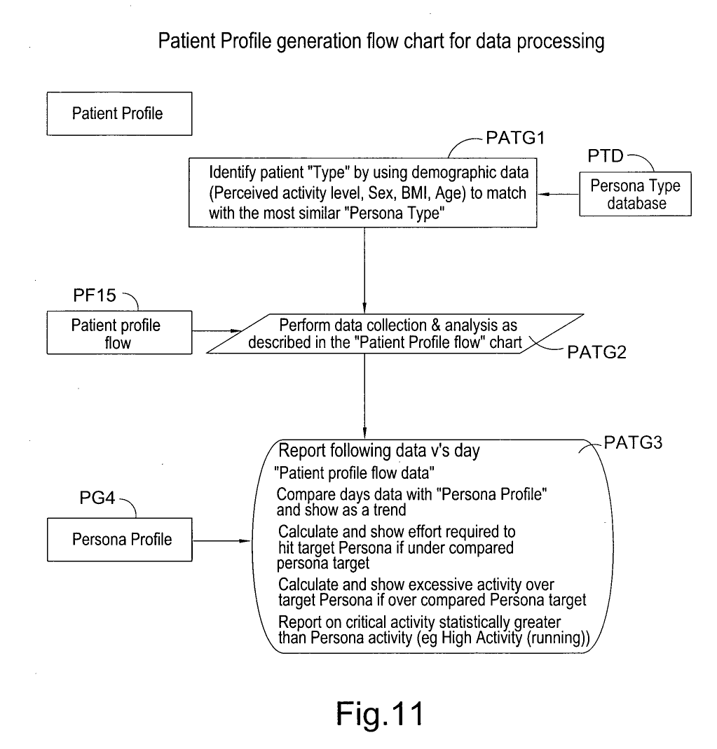

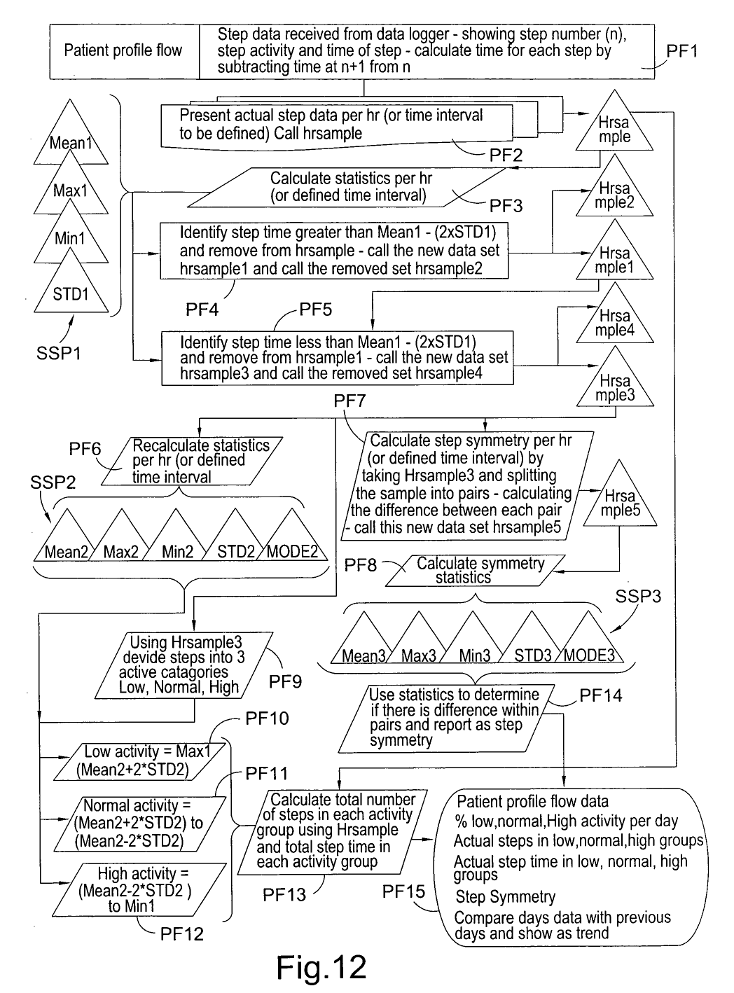

Referring now to FIG. 11, this is a flow chart showing the steps involved in the generation of a patient profile and report used in certain embodiments of the invention. Step PAGT1 identifies the patient type of the particular subject patient being monitored by using demographic data (perceived activity level, sex, BM1, age etc) to match with the most similar persona type stored in the persona type database PTD. Then, having identified the patient or person type of the subject step PATG2 performs data collection and analysis (i.e. motion data collection and analysis, including statistical analysis) as described in the “patient profile flow” chart shown in FIG. 12, and described below. Then, the generated patient profile flow data PF15 is analysed in light of the relevant persona PG4 (in other words the patient profile flow data PF15 and persona profile PG4 of that patient type are processed using suitable processed means and a suitable algorithm or algorithms). The result of this processing/analysis is the generation of a report PATG3 which contains a report of the following data verses day (the following information is presented for each day of a series of one or more odd days in the report): the patient profile flow data PF15. The report may also include a comparison of each days data with the relevant persona profile, and an indication of this comparison shown as a trend. The report may also include the results of a calculation to show the effort required to hit a target persona activity level if the observed activity level is under a desired value when compared with a particular persona target. The processing may also have calculated and determined whether there has been any excess activity by the subject when taking into account the target persona activity level. Thus, the report generated in step PATG3 may also include an indication of any excessive activity. The report may also contain information on any critical activity statistically greater than should be expected from the relevant persona profile. Thus, the report can flag up issues such as the patient being involved in excessive high activity, such as running, which would be inappropriate taking into account the medical history of the patient and the statistics of the previous determined persona profiles.

Moving onto FIG. 12, this shows the flow chart or algorithm used in certain embodiments of the invention to generate the patient profile flow data PF15 which is utilised by the method shown in FIG. 11. In this particular example the steps in the patient profile flow chart corresponds to the steps shown in the single persona profile generation method illustrated in FIG. 10 and will not be described again in detail. For example, in step PF1 for the flow chart of FIG. 12, the step data received from the data logger is simply the step data of the monitored subject, as compared with the single persona profile generation method in which step PP1 referred to step data received from one of the people being observed in order to generate the persona profiles. Thus, steps PF2 to PF15 corresponds respectively to steps PP2 to PP15. Statistical parameters SSP1 generated from step PF3 correspond to them statistical parameters SP1 generated from step PP3, parameters SSP2 generated from PF6 correspond to parameters SP2 generated PP6, and parameters SSP3 corresponded to parameters SP3 generated by step PPB. Finally, the result of the method shown in FIG. 12 is a set of processed patient profile data PF15 which may, for example, contain respective percentages of low, normal and high activity per day, the actual number of steps in the low, normal, and high groups, the actual step time in low, normal and high groups, an indication of step symmetry, and a comparison of the particular days data with a previous days data, those comparison results being shown as a trend. Again, it will be appreciated that these are merely examples and other statistical parameters of the initially observed motion data may be presented in step PF15, such as a simple number indicative of the total number of steps taken per day, per hour, or some other time interval, other statistical parameters could, for example, include a maximum observed step rate in a particular time interval and/or value indicative of step symmetry over a particular time interval.

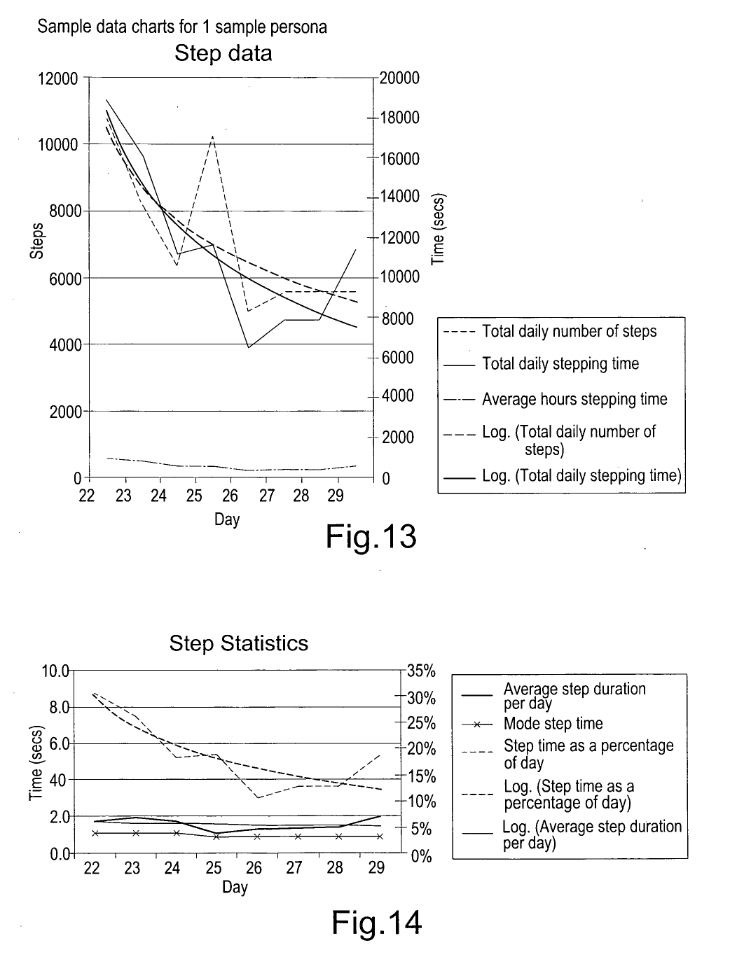

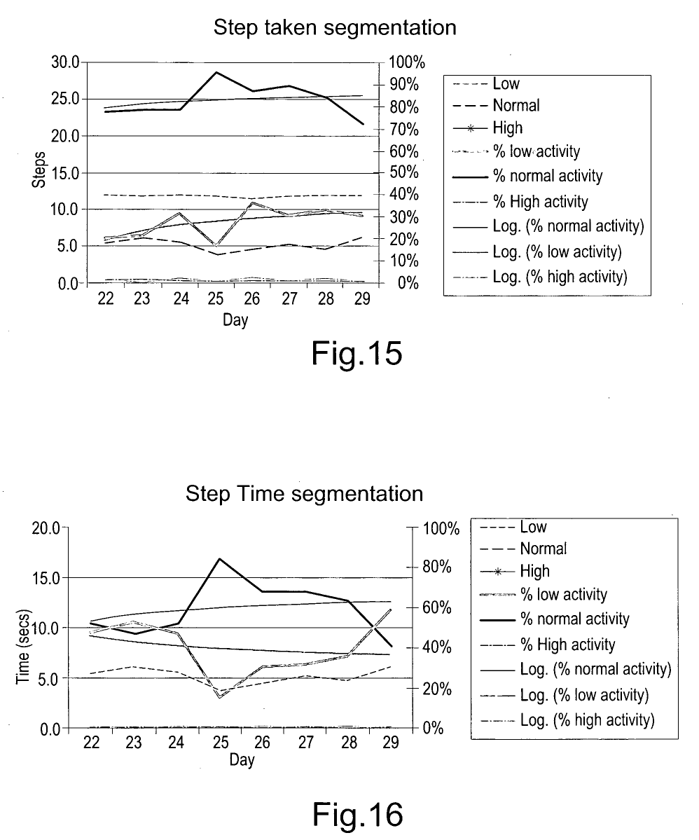

Referring now to FIGS. 13-16, these are examples of plots of step data, steps statistics, steps taken segmentation information, and step time segmentation information as a function of monitoring day generated using monitoring methods embodying the present invention. For example in FIG. 13, one can see that the total daily number of steps taken by the monitored subject is generally declining from day to clay, although the actual number of steps taken in a particular day shows a peak on day 25.

1. A patient monitoring method comprising:
monitoring motion of a subject patient and generating motion data indicative of at least one aspect of the subject's motion;
processing said motion data to generate at least one statistical parameter of said data;
identifying a person type of the subject according to a plurality of characteristics;
accessing a database containing a plurality of stored, predetermined statistical parameters classified according to a plurality of said person types, each stored statistical parameter being a statistical parameter of motion data corresponding to a respective one of said plurality of person types and having been determined by a method comprising processing of motion data obtained by monitoring motion of at least one person having said respective one of said plurality of person types; and
processing at least one said generated statistical parameter and at least one of said stored statistical parameters corresponding to the person type of said subject patient.

2. A method in accordance with claim 1, wherein said processing at least one said generated statistical parameter and at least one of said stored statistical parameters corresponding to the person type of said subject patient comprises processing at least one said generated statistical parameter and at least one of said stored statistical parameters corresponding to the person type of said subject patient to determine a course of action for at least one of the subject and a medical practitioner.

3. A method in accordance with claim 1, wherein said monitoring motion of a subject patient comprises providing the subject with motion monitoring means to be worn or carried by the subject, the motion monitoring means being adapted to generate said motion data when worn or carried by the subject in response to motion of the subject.

4. A method in accordance with claim 3, wherein the motion monitoring means is adapted to detect steps taken by the subject and to generate motion data in response to each detected step.

5. A method in accordance with claim 3 wherein the motion monitoring means further comprises processing means for processing said motion data, and the step of processing said motion data comprises processing motion data with the processing means of the motion monitoring means.

6. A method in accordance with claim 3, further comprising the steps of transmitting motion data or processed motion data from the motion monitoring means and receiving the transmitted data at a remote location.

7. A method in accordance with claim 6, further comprising the steps of transmitting identification data indicative of an identity of the subject and receiving the transmitted identification data at the remote location.

8. A method in accordance with claim 6 wherein the motion monitoring means comprises a mobile telephone, and the step of transmitting comprises transmitting data from said mobile telephone.

9. A method in accordance with claim 1, wherein the generated at least one statistical parameter comprises at least one statistical parameter indicative of a respective one of the following, each associated with an aspect of the subject's motion over a predetermined time interval: a mean value; a maximum value; a minimum value; a standard deviation; and a mode value.

10. A method in accordance with claim 1, wherein said motion data comprises motion data indicative of times at which the subject takes a step.

11. A method in accordance with claim 10, wherein the generated at least one statistical parameter comprises at least one statistical parameter indicative of a respective one of the following: a number of steps in a predetermined time interval; a mean number of steps in a predetermined time interval; a maximum number of steps in a predetermined time interval; a minimum number of steps in a predetermined time interval; a standard deviation of a number of steps in a predetermined time interval; a mode value of a number of steps in a predetermined time interval; a step symmetry; a mean step symmetry in a predetermined time interval; a maximum value of step symmetry in a predetermined time interval; a minimum value of step symmetry in a predetermined time interval; a standard deviation of step symmetry value in a predetermined time interval; a mode value of step symmetry in a predetermined time interval; a respective proportion of a predetermined time interval in which a step activity of the subject falls into at least one predetermined category; a mean step interval in a predetermined time interval; a maximum step interval in a predetermined time interval; a minimum step interval in a predetermined time interval; a standard deviation of step interval in a predetermined time interval; and a mode value of step interval in a predetermined time interval.

12. A method in accordance with claim 1, wherein said plurality of characteristics comprises at least two of: sex; age; body mass index;
perceived activity level type; and surgical history.

13. A method in accordance with claim 1, further comprising providing a characteristics database containing data indicative of said subject's identity and data indicative of said plurality of characteristics of the subject, and said identifying a person type of the subject comprises accessing said characteristics database.

14. A method in accordance with claim 1, wherein the step of processing at least one said generated statistical parameter and at least one of said stored statistical parameters comprises comparing at least one generated statistical parameter with a corresponding stored statistical parameter.

15. A method in accordance with claim 1, wherein the step of processing at least one said generated statistical parameter and at least one of said stored statistical parameters comprises using a computer-implemented algorithm.

16. A method in accordance with claim 1, wherein the step of processing at least one said generated statistical parameter and at least one of said stored statistical parameters comprises determining a cost associated with the subject patient according to the generated at least one statistical parameter, determining an expected cost associated with the person type of said subject according to the corresponding stored statistical parameter or parameters, determining a cost associated with an action, and determining a course of action according to said costs.

17. A method in accordance with claim 2, wherein the course of action comprises at least one action for the subject.

18. A method in accordance with claim 17, wherein said at least one action for the subject comprises a change in motion activity.

19. A method in accordance with claim 17, further comprising providing a signal to the subject, recommending said at least one action for the subject.

20. A method in accordance with claim 19, wherein the step of providing a signal to the subject comprises transmitting a signal.

21. A method in accordance with claim 19, wherein the step of providing the signal to the subject comprises making the signal available for access by the subject.

22. A method in accordance with claim 2, wherein the course of action comprises at least one action for a medical practitioner.

23. A method in accordance with claim 22, wherein said at least one action for the medical practitioner comprises: surgery to be performed on the subject, visiting the subject, admission of the subject to a medical facility, or discharging the subject from a medical facility.

24. (canceled)

25. (canceled)

26. (canceled)

27. A method in accordance with claim 22, further comprising providing a signal to the medical practitioner, recommending said at least one action for the medical practitioner.

28. A method in accordance with claim 27, wherein the step of providing a signal to the medical practitioner comprises transmitting the signal to the practitioner.

29. A method in accordance with claim 27, wherein the step of providing the signal to the medical practitioner comprises making the signal available for access by the medical practitioner.

30. A method in accordance with claim 1, further comprising using at least one of the generated motion data and the generated at least one statistical parameter of the subject patient to update said database by modifying at least one stored statistical parameter corresponding to the person type of the subject.

31. A method in accordance with claim 1, further comprising populating said database with the plurality of stored, predetermined statistical parameters using a method comprising: for each person type, identifying at least one person having that person type, monitoring motion of the identified at least one person and generating motion data indicative of at least one aspect of the motion of the at least one identified person, processing the motion data from the at least one identified person to generate at least one statistical parameter of that data, and storing the generated at least one statistical parameter of the motion data of the at least one identified person in the database, classified according to person type of the at least one identified person.

32. A method in accordance with claim 31, wherein the step of identifying at least one person for at least one of the plurality of said person types comprises identifying a plurality of persons having that person type.

33. A patient monitoring system comprising:
motion monitoring means adapted to monitor motion of a subject patient and generate motion data indicative of at least one aspect of the subject's motion;
processing means arranged to process said motion data to generate at least one statistical parameter of said data;
identification means arranged to identify a person type of the subject according to a plurality of characteristics;
a database containing a plurality of stored, predetermined statistical parameters classified according to a plurality of said person types, each stored statistical parameter being an expected statistical parameter of motion data corresponding to a respective one of said plurality of person types; and
processing means arranged to process at least one said generated statistical parameter and at least one of said stored statistical parameters corresponding to the person type of said subject patient.

34. A system in accordance with claim 33, wherein the processing means arranged to process at least one said generated statistical parameter and at least one of said stored statistical parameters corresponding to the person type of said subject patient is further arranged to determine a course of action for at least one of the subject and a medical practitioner according to the processed statistical parameters.

35. A system in accordance with claim 33, wherein said motion monitoring means is adapted to be worn or carried by the subject, and is adapted to generate said motion data, when worn or carried by the subject, in response to motion of the subject.

36. A system in accordance with claim 35, wherein the motion monitoring means is adapted to detect steps taken by the subject and to generate motion data in response to each detected step.

37. A system in accordance with claim 35 wherein the motion monitoring means further comprises processing means adapted to process said motion data.

38. A system in accordance with claim 35, wherein the motion monitoring means further comprises transmitting means adapted to transmit motion data or processed motion data for reception at a remote location and the system further comprises receiving means arranged to receive the transmitted data at the remote location.

39. A system in accordance with claim 38, wherein the motion monitoring means is further adapted to store identification data indicative of an identity of the subject, and the transmitting means is adapted to transmit said identification data for reception at the remote location.

40. A system in accordance with claim 38 wherein the transmitting means is adapted to transmit data in a wireless signal.

41. A system in accordance with claim 34, wherein the generated at least one statistical parameter comprises at least one statistical parameter indicative of a respective one of the following, each associated with an aspect of the subject's motion over a predetermined time interval: a mean value; a maximum value; a minimum value; a standard deviation; and a mode value.

42. A system in accordance with claim 34, wherein said motion data comprises motion data indicative of times at which the subject takes a step.

43. A system in accordance with claim 34, further comprising a characteristics database containing data indicative of said subject's identity and data indicative of said plurality of characteristics of the subject.

44. A system in accordance with claim 43, further comprising means for inputting identity data and characteristics data into the characteristics database.

45. A system in accordance with claim 43, wherein said plurality of characteristics comprises at least two of: sex; age; body mass index;
perceived activity level type; and surgical history.

46. A system in accordance with claim 34, wherein the processing means arranged to process at least one said generated statistical parameter and at least one of said stored statistical parameters is adapted to compare at least one generated statistical parameter with a corresponding stored statistical parameter.

47. A system in accordance with claim 34, wherein the processing means arranged to process at least one said generated statistical parameter and at least one of said stored statistical parameters is arranged to process said parameters using a predetermined algorithm.

48. A system in accordance with claim 34, wherein the processing means arranged to process at least one said generated statistical parameter and at least one of said stored statistical parameters is arranged to determine a course of action for at least one of the subject and a medical practitioner according to the processed statistical parameters, and is arranged to determine a cost associated with the subject patient according to the generated at least one statistical parameter, determine an expected cost associated with the person type of said subject according to the corresponding stored statistical parameter or parameters, determine a cost associated with an action, and determine a course of action according to said costs.

49. A system in accordance with claim 34, wherein the processing means arranged to process at least one said generated statistical parameter and at least one of said stored statistical parameters is arranged to determine a course of action for at least one of the subject and a medical practitioner according to the processed statistical parameters, and the course of action comprises at least one action for the subject.

50. A system in accordance with claim 49, wherein said at least one action for the subject comprises a change in motion activity.

51. A system in accordance with claim 49, wherein the system further comprises signalling means adapted to provide a signal to the subject, the signal recommending said at least one action for the subject.

52. A system in accordance with claim 34, wherein the processing means arranged to process at least one said generated statistical parameter and at least one of said stored statistical parameters is arranged to determine a course of action for at least one of the subject and a medical practitioner according to the processed statistical parameters, and the course of action comprises at least one action for a medical practitioner.

53. A system in accordance with claim 52, wherein the system further comprises signalling means adapted to provide a signal to a medical practitioner, recommending said at least one action for the medical practitioner.

54. A system in accordance with claim 34, further comprising processing means adapted to update said database by modifying at least one stored statistical parameter corresponding to the person type of the subject using at least one of the generated motion data and the generated at least one statistical parameter.

55. (canceled)

56. (canceled)
